(19) United States
(12) Patent Application Publication
(10) Pub. No.: US 2008/0193390 A1
(43) Pub. Date: Aug. 14, 2008

(54) Chemical Composition and Process to Obtaining of Remover Product of Carious Tissue and Application of the Product

(76) Inventors: Marcia Boucas, Sao Paulo (BR); Sandra Kalil Bussadori, Sao Paulo (BR)

Correspondence Address: Pearl Cohen Zedek Latzer, LLP, 1500 Broadway, 12th Floor, New York, NY, 10036, US

(21) Appl. No.: 10/569,724

(22) PCT Filed: Aug. 26, 2004
(86) PCT No.: PCT/BR2004/000159

§ 371 Date: Mar. 13, 2008

(30) Foreign Application Priority Data
Aug. 28, 2003 (US) PI 0303435-6

(51) Int. Cl. A61K 8/66; A61Q 11/00

(52) U.S. Cl. 424/50

(57) ABSTRACT

Chemical composition and process to obtaining of remover product of carious tissue and application of the product formed from a base of Methylparaben solubilized in hot distilled water, in which it's added Papain, Chloramine, Sodium Chloride and Colorant, adding the Sodium Hydroxide little by little and, finally, the composition of thickener Carboxymethylcellulose to obtain a kind of Gel applied on caries and, after predetermined waiting period, causing the softening of the contaminated tissue which can be removed by simple curettage, after operating procedures in dentistry.

The present descriptive report refers to a patent application to a composition of a kind of “gel”, and its use, after the operating procedures to caries removal in dentistry, and the obtained product is applied on the caries to, after a predetermined waiting time, with the softening of the contaminated tissue, effect the simple curettage and washing of the surface, without necessity of using anesthesia and motorized dental drill.

### PRIOR ART

It's known, principally by dental surgeons, during the operating procedures to treatment and extraction of caries, it's formed a contaminated tissue which has to be removed. Therefore, the dental surgeon has to utilize the motorized dental drill and to apply anesthesia, that can causing expenses, besides discomfort to the patient.

### OBJECTIVE OF THE PATENT

To solve these problems, it was developed the product at issue, whose Methylparaben-based composition is solubilized in hot water, in which it's added Papain, Chloramine, Sodium Chloride and Colorants, adding the Sodium Hydroxide little by little and, finally, the composition of thickener Carboxymethylcellulose, to form the gel.

The product is applied in the infected area, or on the caries and, after waiting about 45 minutes, with the softening of the contaminated tissue, to effect the curettage in the direction of the vestibule to lingual/palatal or mesial and distal, then, the area is washed with water.

Superficially explained, the process of obtaining of the product and its application are more detailed.

The “CHEMICAL COMPOSITION AND PROCESS TO OBTAINING OF REMOVER PRODUCT OF CARIOUS TISSUE AND APPLICATION OF THE PRODUCT.”, object of this present patent application consist of: Papain 6000 Ui/mg 10%; Chloramine T 0.5%; Carboxymethylcellulose 1.5%; Sodium Chloride 0.9%; Methylparaben 0.15%; Sodium Hydroxide 0.2%; Blue Colorant sufficient quantity; Yellow Colorant sufficient quantity; Distilled water 100%.

According the above-mentioned, the product is obtained from a base of Methylparaben solubilized in hot distilled water; there is a predetermined cooling period of the initial solution which receives, after this cooling period, addition of Papain 6000 Ui/mg 10%, Chloramine T 0.5%. Sodium Chloride 0.2% and the blue and yellow colorants (sufficient quantity).

Sodium Hydroxide 0.2% is added gradually, until to set pH of the solution, which has to stay between 6.5 and 7.0, and then, thickener Carboxymethylcellulose 1.5% is added and the obtained solution is shaken until to form a Gel.

Thus compound, the product or Gel, which is based in Papain and Chloramine, will be applied after the isolation of the field of operation, without the need of anesthetize the area or the caries. After waiting 45 minutes, the dental surgeon provided with curette number 17 or 18, utilizing the non-cutting part of the instrument, effects the curettage of the contaminated tissue that it has already softened by the gel's action, in the direction of the vestibule to lingual/palatal or mesial and distal.

In the case of the contaminated tissue don't be removed completely, the Gel is applied again, following an identical procedure to remove the remaining caries and, finally, the area is washed with water.

1-2. (canceled)

3. A composition for removing dental carries, comprising: 10% Papain 6000 Ui/mg; 0.5% Chloramine T; 1.5% Carboxymethylcellulose; 9% Sodium Chloride; 0.15% Methylparaben; 0.2% Sodium Hydroxide; and distilled water, wherein said composition is in the form of a gel.

4. A method of removing dental carries comprising the steps of: isolating the field of operation; applying the composition of claim 3 onto the dental carries; allowing the gel to soften the dental carries; and curetting the carries using a non-cutting part of a curette, in the direction of the vestibule towards the lingual/palatial, mesial or distal direction, wherein the steps above are repeated in case dental carries remain in the field of operation, thereby removing dental carries.

5. The method of claim 4, comprising waiting about 45 minutes between application of the gel and curettage.

6. The composition of claim 3, prepared by the process comprising the steps of: solubilizing Methylparaben in hot distilled water, creating an initial solution; cooling the initial solution; adding 10% Papain 6000 Ui/mg, 0.5% Chloramine T 0, 0.2% sodium chloride and blue and yellow colorants, creating an admixture; gradually adding Sodium Hydroxide until the pH of the admixture is between 6.5 to about 7.0; adding Carboxymethylcellulose; and shaking the thickened, pH-adjusted admixture until a gel is formed.
